(19) United States
(12) Patent Application Publication
(10) Pub. No.: US 2026/0256659 A1
(43) Pub. Date: Sep. 3, 2026

(54) COMPLEX FOR SEALING DENTINAL TUBULE, REAGENT FOR TREATING DENTIN HYPERSENSITIVITY, AND USE THEREOF

(71) Applicant: ANHUI MEDICAL UNIVERSITY, Hefei (CN)

(72) Inventors: Xiangyang LI, Hefei (CN); Yuan LI, Hefei (CN); Wenjun WANG, Hefei (CN); Jialong CHEN, Hefei (CN); Hua QIU, Hefei (CN); Weirui NIU, Hefei (CN); Liuwei HUANG, Hefei (CN)

(73) Assignee: ANHUI MEDICAL UNIVERSITY, Hefei (CN)

(21) Appl. No.: 19/489,915

(22) PCT Filed: Dec. 30, 2024
(86) PCT No.: PCT/CN2024/143670

§ 371 Date: Dec. 4, 2025

(30) Foreign Application Priority Data
Oct. 30, 2023 (CN) 202311412278.5

(51) Int. Cl. A61K 6/20; A61K 6/60; A61K 6/70; A61K 8/19; A61K 31/05; A61K 33/24; A61P 1/02; A61Q 11/00

(52) U.S. Cl. CPC A61K 6/20; A61K 6/60; A61K 6/70; A61K 8/19; A61K 31/05; A61K 33/24; A61P 1/02; A61Q 11/00

(57) ABSTRACT

Provided are a complex for sealing a dentinal tubule (DT), a reagent for treating dentin hypersensitivity (DH), and use thereof. The reagent for treating DH includes the complex for sealing the DT, the complex being produced through polymerization of a catecholamine with metal ions. The catecholamine includes at least one of dopamine, norepinephrine, tannic acid, gallic acid, phlorotannin, phloroglucinol monomer, and epigallocatechin gallate; and the metal ions include at least one of V²⁺ ions, Cr²⁺ ions, Cr³⁺ ions, Mn²⁺ ions, Mn³⁺ ions, Fe²⁺ ions, Fe³⁺ ions, Co²⁺ ions, Ni²⁺ ions, Cu²⁺ ions, and Sr²⁺ ions.

### CROSS REFERENCE TO THE RELATED APPLICATIONS

This application is the national phase entry of International Application No. PCT/CN 2024/143670, filed on Dec. 30, 2024, which is based upon and claims priority to Chinese Patent Application No. 202311412278.5, filed on Oct. 30, 2023, the entire contents of which are incorporated herein by reference.

### TECHNICAL FIELD

The present disclosure belongs to the technical field of oral care, and specifically relates to a complex for sealing a dentinal tubule (DT), a reagent for treating dentin hypersensitivity (DH), and use thereof.

### BACKGROUND

Dentin hypersensitivity (DH) is specifically manifested as severe pain. This pain is caused by the responses of DTs in exposed dentin to heat, evaporation, tactile osmosis, and chemical and electrical stimulation. Therefore, the sealing of exposed DTs is the key to reducing or eliminating DH.

Currently, the main sealing materials used in clinical practice are sealing resins, dentin bonding agents, remineralization coatings, and other coatings for clinical practice. However, most of the sealing materials cannot achieve satisfactory results, which is attributed to the following reasons: on one hand, a sealing material is easy to fall off due to mechanical friction; on the other hand, the unpolymerized monomers leached from the adhered smear layers (MERs) have adverse reactions, such as changes in hypersensitivity, estrogenic activity, and immune response. In addition, the oral bacteria easily attach to the salivary acquired pellicle (SAP) to form a biofilm, produce toxins, and even cause chronic diseases such as pulpitis. However, the antibacterial properties of desensitization products have long been neglected. Therefore, how to achieve the deep sealing of DTs while allowing anti-bacteria and remineralization and maintaining stable properties of DTs is crucial for the long-term treatment of DH.

### SUMMARY

An object of the present disclosure is to provide a complex for sealing DT, a reagent for treating DH, and use thereof. The complex for sealing DT provided in the present disclosure could improve the DH. The complex could allow for the rapid and efficient sealing of DTs, has an antibacterial ability for caries pathogens, and exhibits excellent biocompatibility.

To achieve the above object, the present disclosure provides the following technical solutions:

The present disclosure provides a complex for sealing a DT, where the complex is produced through polymerization of a catecholamine with metal ions.

In some embodiments, the catecholamine includes one or more selected from the group consisting of dopamine, norepinephrine, tannic acid, gallic acid, phlorotannin (PL), phloroglucinol monomer, and epigallocatechin gallate.

In some embodiments, the metal ions include one or more selected from the group consisting of V²⁺ ions, Cr²⁺ ions, Cr³⁺ ions, Mn²⁺ ions, Mn³⁺ ions, Fe²⁺ ions, Fe³⁺ ions, Co²⁺ ions, Ni²⁺ ions, Cu²⁺ ions, and Sr²⁺ ions.

The present disclosure also provides a method for preparing the complex for sealing the DT described in the above solutions, including the following step: under aerobic and alkaline conditions, mixing the catecholamine and the metal ions to obtain a mixed solution, and subjecting the mixed solution to a reaction to produce the complex for sealing the DT.

In some embodiments, the reaction is conducted for 1 min to 30 min.

In some embodiments, the reaction is conducted for 1 min to 5 min.

In some embodiments, the reaction is conducted for 5 min to 10 min.

In some embodiments, the reaction is conducted for 10 min to 15 min.

In some embodiments, the reaction is conducted for 15 min to 20 min.

In some embodiments, the reaction is conducted for 20 min to 25 min.

In some embodiments, the reaction is conducted for 25 min to 30 min.

In some embodiments, an alkaline condition refers to a pH value of 7.4 to 11.

In some embodiments, the alkaline condition refers to a pH value of 8.5 to 10.

In some embodiments, the alkaline condition is provided by 1.21 mg/mL Tris buffer.

In some embodiments, the aerobic condition is provided by exposing to air.

In some embodiments, in the mixed solution obtained by mixing the catecholamine and the metal ions, a concentration of the PL is in a range of 0.1 mg/mL to 5 mg/mL.

In some embodiments, the concentration of the PL is in a range of 0.1 mg/mL to 0.4 mg/mL.

In some embodiments, the concentration of the PL is in a range of 0.4 mg/mL to 0.8 mg/mL.

In some embodiments, the concentration of the PL is in a range of 0.8 mg/mL to 1.2 mg/mL.

In some embodiments, the concentration of the PL is in a range of 1.2 mg/mL to 1.6 mg/mL.

In some embodiments, the concentration of the PL is in a range of 1.6 mg/mL to 5 mg/mL.

In some embodiments, a concentration of the Sr²⁺ ions is in a range of 0.1 mg/mL to 0.4 mg/mL.

In some embodiments, the concentration of the Sr²⁺ ions is in a range of 0.4 mg/mL to 0.8 mg/mL.

In some embodiments, the concentration of the Sr²⁺ ions is in a range of 0.8 mg/mL to 1.2 mg/mL.

In some embodiments, the concentration of the Sr²⁺ ions is in a range of 1.2 mg/ml to 1.6 mg/mL.

In some embodiments, the concentration of the Sr²⁺ ions is in a range of 1.6 mg/mL to 5 mg/mL.

In some embodiments, the concentration of the PL is 0.4 mg/mL and the concentration of the Sr²⁺ ions is 1.6 mg/mL.

The present disclosure also provides a reagent for treating DH, including the complex for sealing the DT described in the above solutions or the complex for sealing the DT prepared by the method described in the above solutions.

In some embodiments, the reagent for treating DH further includes: an alkaline amino acid in a free or salt form, a fluoride salt, an anti-inflammatory compound, an antilithic agent, a surfactant, a humectant, an antibacterial agent, and an antioxidant, each in an effective amount.

In some embodiments, the reagent for treating DH is selected from the group consisting of a toothpaste, a mouthwash, and a drug.

The present disclosure also provides use of the reagent for treating DH described in the above solutions in preparation of a drug for treating DH, a drug for treating dental caries, or a drug for treating an enamel damage.

The present disclosure also provides a toothpaste, including the reagent for treating DH described in the above solutions, and further including one or more selected from the group consisting of water, an abrasive, a surfactant, a foaming agent, a vitamin, a polymer, an enzyme, a humectant, a thickening agent, an antibacterial agent, a preservative, a flavoring agent, and a coloring agent.

The present disclosure provides a complex for sealing DT. The complex provided in the present disclosure could seal nearly 85% of DTs within 3 min, and could form a similar crystal structure to hydroxyapatite after being mineralized in vitro for 7 days. Results of a 7-day SD rat animal experiment are consistent with the results of the in vitro mineralization experiment. In addition, the complex could be prepared rapidly, and exhibits an antibacterial ability against caries pathogens and excellent biocompatibility.

### BRIEF DESCRIPTION OF THE DRAWINGS

FIG. 1A to FIG. 1P show scanning electron microscopy (SEM) images for the sealing of DTs with PL and strontium chloride at different concentrations;

FIG. 2A to FIG. 2E show field emission scanning electron microscopy images of dentin sections soaked in distilled water, a PL solution, a strontium chloride solution, a PL@Sr solution, and a phloroglucinol@Sr solution, and FIG. 2F shows an SEM image of contents of DTs in a PL@Sr-treated dentin section after a negative pressure suction treatment;

FIG. 3 shows sealing rates of dentin sections treated with PL, dentin sections treated with Sr, and dentin sections treated with PL@Sr;

FIG. 4 shows stability results of a PL@Sr sealing material soaked in normal saline for different times;

FIG. 5A to FIG. 5D show mineralization results of dentin sections treated with PL@Sr, where FIG. 5A shows SEM images of longitudinal sections in four groups of dentin after 4 h and 24 h of remineralization; FIG. 5B shows SEM images of transverse sections in four groups of dentin after 4 h and 24 h of remineralization; FIG. 5C shows an X-ray diffraction (XRD) pattern of the four groups of dentin after 4 h of remineralization; and FIG. 5D shows an XRD pattern of the four groups of dentin after 24 h of remineralization;

FIG. 6A to FIG. 6E show acid corrosion resistance results of dentin sections treated with PL@Sr and mineralized, where FIG. 6A to FIG. 6C show SEM images of normal dentin, dentin sealed with PL@Sr, and dentin sealed and then mineralized for 24 h after corrosion in 5 wt % citric acid for 90 s, respectively; FIG. 6D and FIG. 6E show concentration losses of calcium (Ca) and phosphorus (P) in normal dentin, dentin sealed with PL@Sr, and dentin sealed and then mineralized for 24 h after corrosion in 5 wt % (n=3) citric acid for 90 s, respectively; and a significant difference is indicated through one-way analysis of variance (P<0.05);

FIG. 7A to FIG. 7H show frictional wear experiment results of dentin sections treated with PL@Sr and mineralized, where FIG. 7A to FIG. 7F show SEM images of wear traces on normal dentin, dentin sealed with PL@Sr, and dentin sealed and mineralized for 24 h (hereinafter referred to as a mineralization group); FIG. 7G shows coefficients of friction and wearing depths (n=3), and a difference between groups is statistically significant (p<0.05); and FIG. 7H shows Young's hardness (n=5), and a difference between groups is statistically significant (p<0.05);

FIG. 8A and FIG. 8B show biocompatibility results of dentin sections treated with PL@Sr, where FIG. 8A shows rhodamine fluorescence staining results of a blank group, a PL group, a Sr group, and a PL@Sr group after being co-cultured with L929 for 1 d, 3 d, and 5 d, and FIG. 8B shows cell proliferation-toxicity (CCK-8) results of the four groups of dentin after being co-cultured with L929 for 1 d, 3 d, and 5 d;

FIG. 9A and FIG. 9B show antibacterial results of dentin sections sealed with PL@Sr, where FIG. 9A shows bacterial absorbance values of a blank group, a PL group, a Sr group, and a PL@Sr group after being incubated with Streptococcus mutans for 4 h (n=3, p<0.05); FIG. 9B shows bacterial number counting in the blank group, the PL group, the Sr group, and the PL@Sr group; and

FIG. 10A to FIG. 10D show results of an animal experiment, i.e., surface morphologies of dentin in a blank group and a PL@Sr group on day 1 and day 7, respectively.

### DETAILED DESCRIPTION OF THE EMBODIMENTS

The present disclosure provides a reagent for treating DH, including a complex for sealing DT.

In some embodiments, the reagent for treating DH provided in the present disclosure further includes:
i. an effective amount of an alkaline amino acid in a free or salt form; and
ii. optionally, an effective amount of a fluoride, where the fluoride is preferably a soluble fluoride salt.

In some embodiments of the present disclosure, the alkaline amino acid is selected from the group consisting of arginine, lysine, citrulline, ornithine, creatine, histidine, diaminobutyric acid, diaminopropionic acid, salts of the above amino acids, and/or combinations thereof.

In some embodiments of the present disclosure, the alkaline amino acid has an L configuration.

In some embodiments of the present disclosure, the alkaline amino acid is arginine; in some embodiments, the arginine is L-arginine.

In some embodiments of the present disclosure, the alkaline amino acid is partially or completely in a salt form; in some embodiments, a salt of the alkaline amino acid includes one or more selected from the group consisting of arginine phosphate, arginine hydrochloride, arginine sulfate, and arginine bicarbonate.

In some embodiments of the present disclosure, the salt of the alkaline amino acid is produced in situ in the reagent by neutralizing the alkaline amino acid with an acid or a salt of the acid. In some embodiments, the salt of the alkaline amino acid is produced by neutralizing the alkaline amino acid to produce a premix before binding to the soluble fluoride salt.

In some embodiments of the present disclosure, the fluoride salt is one or more selected from the group consisting of stannous fluoride, sodium fluoride, potassium fluoride, sodium monofluorophosphate, sodium fluorosilicate, ammonium fluorosilicate, amine fluoride (the amine fluoride is preferably N′-octadecyltrimethylenediamine-N,N,N′-tris(2-ethanol)-dihydrofluoride), ammonium fluoride, titanium fluoride, and a hexafluorosulfate salt.

In some embodiments of the present disclosure, the fluoride salt is a fluorophosphate salt.

In some embodiments of the present disclosure, the fluoride salt is sodium monofluorophosphate.

In some embodiments, the reagent for treating DH provided in the present disclosure further includes one or more selected from the group consisting of an abrasive and microparticles.

In some embodiments of the present disclosure, the microparticles are one or more selected from the group consisting of sodium bicarbonate, calcium phosphate (the calcium phosphate is preferably dicalcium phosphate dihydrate), calcium sulfate, precipitated calcium carbonate, silica (the silica is preferably hydrated silica), iron oxide, alumina, perlite, and plastic particles (the plastic particles are preferably polyethylene particles).

In some embodiments of the present disclosure, the abrasive is one or more selected from the group consisting of calcium phosphate (the calcium phosphate is preferably dicalcium phosphate dihydrate), calcium sulfate, precipitated calcium carbonate, silica (the silica is preferably hydrated silica), and calcium pyrophosphate.

In some embodiments, the reagent for treating DH provided in the present disclosure further includes an antilithic agent.

In some embodiments of the present disclosure, the antilithic agent is a polyphosphate salt, and the polyphosphate salt is preferably selected from the group consisting of a pyrophosphate salt, a tripolyphosphate salt, and a hexametaphosphate salt. In some embodiments, the polyphosphate salt is a sodium salt.

In some embodiments, the reagent for treating DH provided in the present disclosure further includes a surfactant.

In some embodiments of the present disclosure, the surfactant is one or two selected from the group consisting of sodium lauryl sulfate and cocamidopropyl betaine.

In some embodiments, the reagent for treating DH provided in the present disclosure further includes an anionic surfactant.

In some embodiments of the present disclosure, the anionic surfactant is sodium lauryl sulfate.

In some embodiments, the reagent for treating DH provided in the present disclosure further includes a humectant. In some embodiments, the humectant is one or more selected from the group consisting of glycerol and sorbitol.

In some embodiments, the reagent for treating DH provided in the present disclosure further includes a polymer. In some embodiments, the polymer is one or more selected from the group consisting of polyethylene glycol, a polymethyl vinyl ether-maleic acid copolymer, and a polysaccharide (the polysaccharide is preferably a cellulose derivative or a polysaccharide gum, where the cellulose derivative is preferably carboxymethyl cellulose and the polysaccharide gum is preferably xanthan gum or carrageenan).

In some embodiments, the reagent for treating DH provided in the present disclosure further includes an antibacterial agent. In some embodiments, the antibacterial agent is one or more selected from the group consisting of a halogenated diphenyl ether (the halogenated diphenyl ether is preferably triclosan), a herbal plant extract and essential oil (the herbal plant extract and essential oil preferably includes a rosemary extract, a tea extract, a magnolia extract, thymol, menthol, cineol, geraniol, carvacrol, citral, hinokitiol, catechol, methyl salicylate, epigallocatechin gallate, epigallocatechin, gallic acid, a miswak extract, and a seabuckthorn extract), a biguanide antibacterial agent (the biguanide antibacterial agent is preferably selected from the group consisting of chlorhexidine, alexidine, and decamethidine), a quaternary ammonium compound (the quaternary ammonium compound is preferably selected from the group consisting of cepylpyridinium chloride (CPC), benzalkonium chloride, tetradecyl pyridinium chloride (TPC), and tetradecylethyl pyridinium chloride (TDEPC)), a phenol antibacterial agent, hexetidine, decamethidine, sanguinarine, povidone iodine, delmopinol, capryloyl salicylic acid (salifluor), metal ions (the metal ions are preferably selected from the group consisting of a zinc salt, a tin salt, a copper salt, and an iron salt, where the zinc salt is preferably zinc citrate), sanguinarine, propolis, an oxidant (the oxidant is preferably selected from the group consisting of hydrogen peroxide, buffered sodium perborate, and sodium percarbonate), phthalic acid and a salt thereof, monoperoxyphthalic acid and a salt and ester thereof, ascorbyl stearate, oleoyl sarcosine, alkyl sulfate, dioctyl sodium sulfosuccinate, salicylanilide, domiphen bromide, delmopinol, octapinol and other piperidine derivatives, a Saccharococcus thermophilus preparation, and a chlorite salt.

In some embodiments, the reagent for treating DH provided in the present disclosure further includes an anti-inflammatory compound. In some embodiments, the anti-inflammatory compound is an inhibitor for at least one host pro-inflammatory factor. In some embodiments, the host pro-inflammatory factor is one or more selected from the group consisting of matrix metalloproteinase (MMP), cyclooxygenase (COX), PGE2, interleukin 1 (IL-1), IL-1β converting enzyme (ICE), transforming growth factor β1 (TGF-β1), inducible nitric oxide synthase (iNOS), hyaluronidase, cathepsin, nuclear factor kappa B (NF-κβ), and IL-1 receptor-associated kinase (IRAK). In some embodiments, the anti-inflammatory compound is one or more selected from the group consisting of aspirin, ketorolac, flurbiprofen, ibuprofen, naproxen, indomethacin, aspirin, ketoprofen, piroxicam, meclofenamic acid, and nordihydroguaiaretic acid.

In some embodiments, the reagent for treating DH provided in the present disclosure further includes an antioxidant. In some embodiments, the antioxidant is one or more selected from the group consisting of coenzymes Q10 and pyrroloquinoline quinone (PQQ), vitamin C, vitamin E, vitamin A, and anethole dithiothione.

In some embodiments, the reagent for treating DH provided in the present disclosure further includes a whitening agent selected from the group consisting of whitening active substances. In some embodiments, the whitening active substances are one or more selected from the group consisting of a peroxide, a metal chlorite, a perborate salt, a percarbonate salt, perhydroxy acid, and a hypochlorite salt.

In some embodiments, the reagent for treating DH provided in the present disclosure further includes hydrogen peroxide or a hydrogen peroxide source. In some embodiments, the hydrogen peroxide source is selected from the group consisting of urea peroxide, a peroxide salt or composite (the peroxide salt is preferably selected from the group consisting of a perphosphate salt, a percarbonate salt, a perborate salt, a persilicate salt, and a persulfate salt; and the composite is preferably selected from the group consisting of calcium perphosphate, sodium perborate, sodium percarbonate, sodium perphosphate, and potassium persulfate), and a hydrogen peroxide-polymer composite. In some embodiments, the hydrogen peroxide-polymer composite is a hydrogen peroxide-polyvinylpyrrolidone polymer composite.

In some embodiments, the reagent for treating DH provided in the present disclosure further includes a calcium and phosphate source. In some embodiments, the calcium and phosphate source is selected from the group consisting of (i) a calcium-glass composite and (ii) a calcium-protein composite. In some embodiments, the calcium-glass composite is calcium sodium phosphosilicate. In some embodiments, the calcium-protein composite is casein phosphopeptide-amorphous calcium phosphate.

In some embodiments, the reagent for treating DH provided in the present disclosure further includes a soluble calcium salt. For example, the soluble calcium salt is one or more selected from the group consisting of calcium sulfate, calcium chloride, calcium nitrate, calcium acetate, and calcium lactate.

In some embodiments, the reagent for treating DH provided in the present disclosure further includes an agent for interfering with or preventing bacterial adhesion, such as ethyl paraben or chitosan.

In some embodiments, the reagent for treating DH provided in the present disclosure further includes a physiologically-acceptable potassium salt, such as potassium nitrate, potassium citrate, or potassium chloride. A specified content of the potassium salt could effectively alleviate the tooth sensitivity.

The present disclosure provides a reagent for treating DH, where in some embodiments, the salt of the alkaline amino acid is arginine bicarbonate, the fluoride is sodium monofluorophosphate, and the abrasive is precipitated calcium carbonate.

The present disclosure provides a reagent for treating DH, where in some embodiments, the salt of the alkaline amino acid is arginine bicarbonate, the fluoride is sodium monofluorophosphate, and the abrasive is silica.

When applied to the oral cavity, for example, by tooth brushing, the reagent for treating DH provided in the present disclosure could effectively (i) reduce or inhibit the generation of dental caries, (ii) mitigate, repair, or inhibit the dangerous damage to tooth enamels, such as damage detected by quantitative light-induced fluorescence (QLF) or electronic caries monitor (ECM), (iii) reduce or inhibit the demineralization and promote the remineralization of teeth, (iv) alleviate the tooth sensitivity, (v) alleviate or inhibit the gingivitis, (vi) promote the oral ulcer or wound healing, (vii) decrease the level of acid-producing bacteria, (viii) increase the relative level of arginine-hydrolyzing bacteria, (ix) inhibit the formation of a biofilm by microorganisms in the oral cavity, (x) increase and/or maintain the pH of dental plaques at a level of at least about 5.5 after sugar attack, (xi) reduce the accumulation of dental plaques, (xii) alleviate the dryness of mouth, (xiii) clean the teeth and oral cavity, (xiv) mitigate the corrosion, (xv) whiten the teeth, and/or (xvi) immunize the teeth against caries-producing bacteria.

In some embodiments, the reagent for treating DH provided in the present disclosure is in a form selected from the group consisting of a mouthwash, a toothpaste, a dental gel, a tooth powder, a non-abrasive gel, a mousse, a foam, an oral spray, a lozenge, an oral tablet, a dental tool, and a pet care product.

In some embodiments of the present disclosure, the reagent for treating DH is a toothpaste.

In some embodiments of the present disclosure, when the reagent for treating DH is a toothpaste, the toothpaste further includes one or more selected from the group consisting of water, an abrasive, a surfactant, a foaming agent, a vitamin, a polymer, an enzyme, a humectant, a thickening agent, an antibacterial agent, a preservative, a flavoring agent, and a coloring agent.

In order to further illustrate the present disclosure, the solutions of the present disclosure are described in detail below in conjunction with accompanying drawings and examples, but these examples should not be understood as a limitation to the protection scope of the present disclosure.

### Comparative Example 1

Phloroglucinol monomer having a polyphenol structure in PL, was adopted as Comparative Example 1.

### Example 1

The natural polyphenol adopted in the present disclosure was PL.

### 1. Preparation of Complexes With Different Concentrations:

Under alkaline and aerobic conditions, different concentrations of PL (0.2 mg/mL, 0.4 mg/mL, 0.8 mg/mL, and 1.6 mg/mL) and different concentrations of SrCl 2 (0.2 mg/mL, 0.4 mg/mL, 0.8 mg/mL, and 1.6 mg/mL) were mixed in a Tris solution (pH=8.5, 1.21 mg/mL) at room temperature (25° C.), and reacted for 3 min to produce 16 complexes.

### 2. Sealing Effects for DTs:

The complexes prepared above each were applied onto a dentin section, and after 3 min, rinsed with deionized water 3 times to produce PL@Sr dentin sections. The PL@Sr dentin sections each were photographed by SEM.

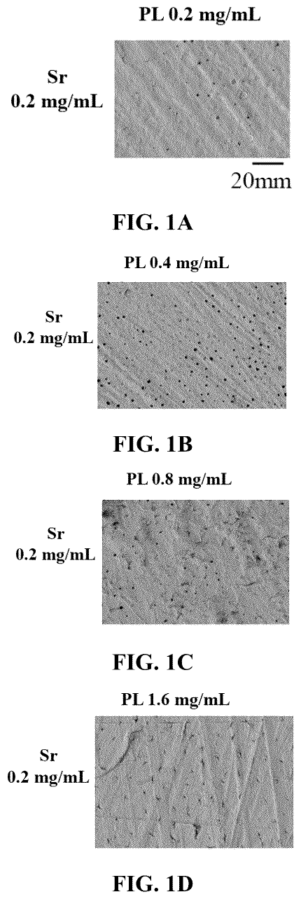

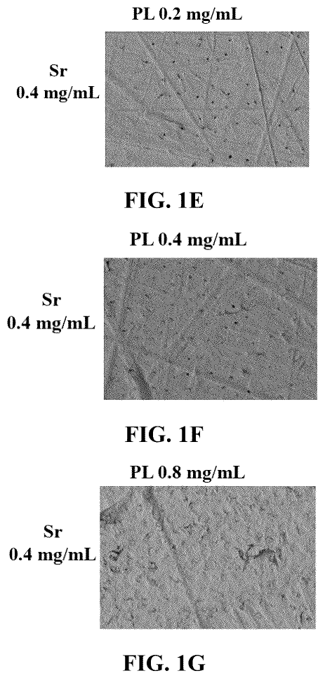

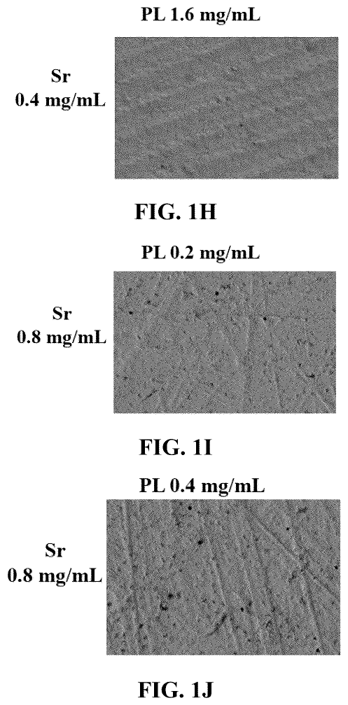

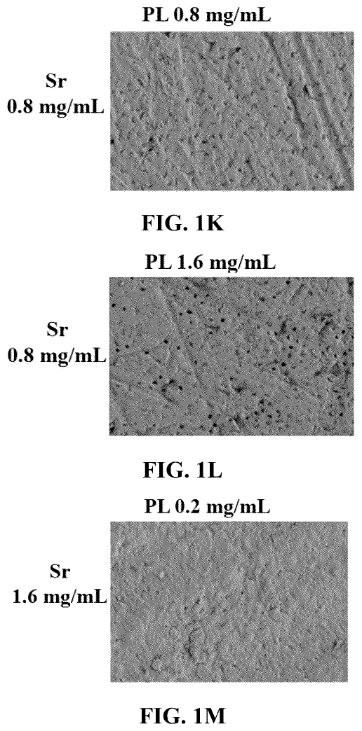

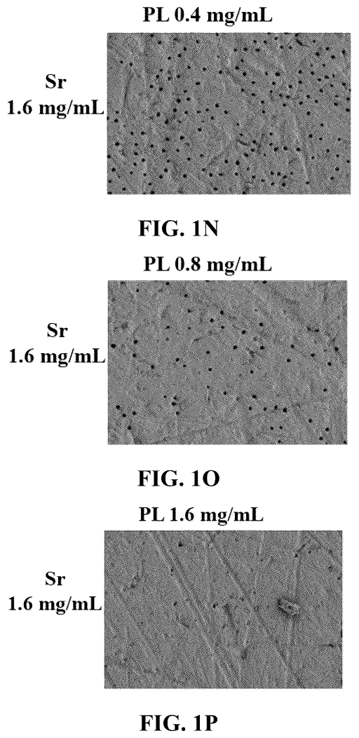

As shown in FIG. 1A to FIG. 1P, a PL@Sr complex with a PL concentration of 0.4 mg/mL and a Sr concentration of 1.6 mg/mL exhibited the optimal effect of sealing DTs.

### Example 2

Investigation of properties of the PL@Sr complex with the optimal concentrations

### 1. Preparation of Samples:

1) Blank control.
2) Under alkaline and aerobic conditions, PL (0.4 mg/mL) and SrCl2 (1.6 mg/mL) were mixed in a Tris solution (pH=8.5, 1.21 mg/mL) at room temperature (25° C.) and reacted for 3 min to produce a test sample. The test sample was applied onto a dentin section, and after 3 min, rinsed with deionized water 3 times to prepare an experimental group.
3) Under alkaline and aerobic conditions, PL (0.4 mg/mL) was subjected to polymerization for 3 min in a Tris solution (pH=8.5, 1.21 mg/mL) at room temperature (25° C.) to produce a control sample. The control sample was applied onto a dentin section, after 3 min, rinsed with deionized water 3 times to prepare a control group.
4) Under alkaline and aerobic conditions, SrCl₂ (1.6 mg/mL) was dissolved in a Tris solution (pH=8.5, 1.21 mg/mL) at room temperature (25° C.) to produce a control sample. The control sample was applied onto a dentin section and then rinsed with deionized water 3 times to prepare a control group.
5) Phloroglucinol monomer having a polyphenol structure in PL, was adopted as a control. A phloroglucinol@Sr complex was prepared with the optimal concentration combination described in the experimental group, applied onto a dentin section, and after 3 min, rinsed with deionized water 3 times to prepare a control group.

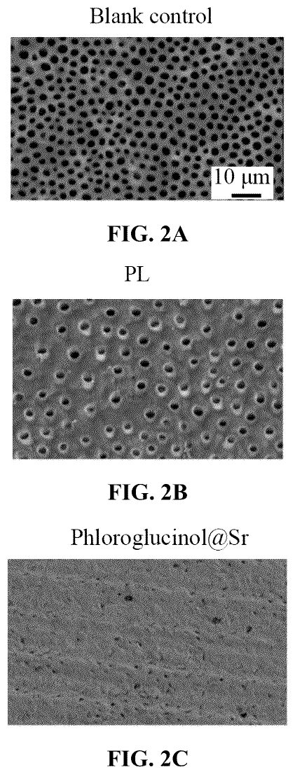

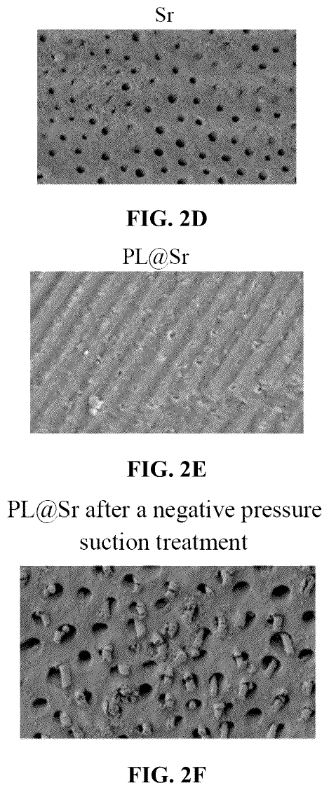

The dentin section samples were separately soaked in distilled water, a PL solution, a phloroglucinol@Sr solution, a strontium chloride solution, and a PL@Sr solution for 3 min, and then photographed by field emission scanning electron microscopy to prepare dentin section images. A dentin section undergoing a PL@Sr treatment was subjected to a negative pressure suction treatment, and the surface morphologies of contents of DTs were photographed by SEM. As shown in FIG. 2A to FIG. 2F, a phloroglucinol@Sr complex has a prominent sealing effect for a dentin section, but the sealing effect is inferior to a sealing effect of PL@Sr. Results of PL@Sr after the negative pressure suction treatment show that the PL@Sr complex still maintains an intact structure under negative pressure. A pore size of the dentin section and the PL@Sr complex sucked out under negative pressure can also indicate the excellent sealing effect of PL@Sr.

### 2. Calculation of Relative Sealing Rates for DTs:

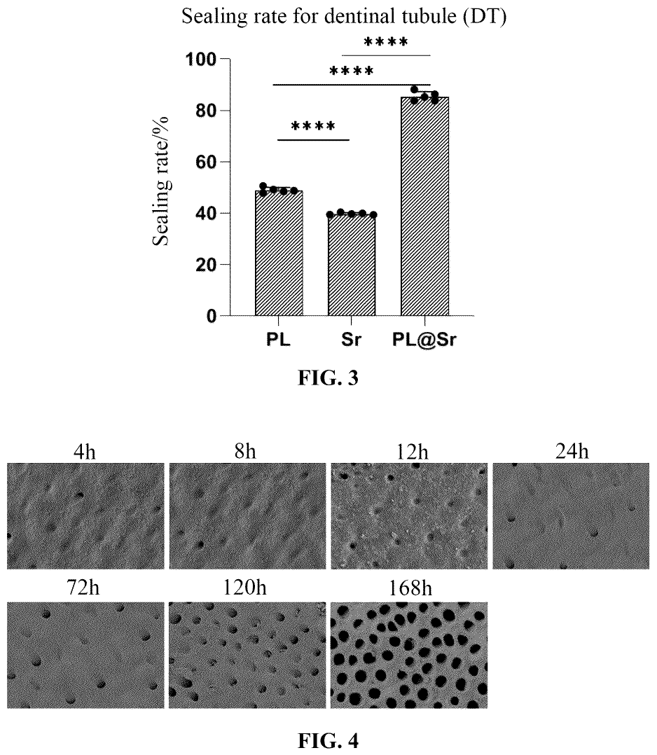

A blank dentin section, a PL dentin section, a Sr dentin section, and a PL@Sr dentin section were selected and observed by SEM. A dentin section was scanned under the 1,000× field of view. An unsealed area of DT was measured with image J. Calculation was conducted according to equation as follows: relative opening rate for DT=opening area for sample DT/sealing area for blank DT×100%, and relative sealing rate for DT=(1-relative opening rate for DT)×100%. Results are shown in FIG. 3.

It can be seen from FIG. 3 that a sealing rate of the PL@Sr complex is as high as 85%, which is much better than the sealing effects of PL and Sr.

### 3. Stability Testing for Sealing Materials:

The dentin samples prepared in the blank group, the PL group, the Sr group, the phloroglucinol@Sr group, and the PL@Sr group were separately placed in a 50 mL clean beaker, and simulated saliva was poured into the beaker for each group. The simulated saliva was changed every 2 days. At 4 h, 8 h, 12 h, 24 h, 72 h, 120 h, and 168 h, each dentin sample was taken out, ultrasonically cleaned with pure water for 30 s, dried, and observed by SEM for surface morphology. Results are shown in FIG. 4.

It can be seen from the SEM surface morphologies of the PL@Sr coating at 4 h, 8 h, 12 h, 24 h, 72 h, 120 h, and 168 h of soaking in normal saline in FIG. 4 that the sealing material is gradually dissolved with the extension of soaking time, and is completely dissolved after 168 h of soaking.

### 4. Dentin Mineralization Experiment:

The dentin sections prepared above were placed at a bottom of a sealed beaker filled with 50 mL of a mineralization solution, and the beaker was placed in a 37° C. incubator. A sample was randomly taken out from each group at regular intervals (4 h and 24 h). All recovered dentin sections were subjected to a gentle ultrasonic treatment in pure water for 2 min, then rinsed with flowing deionized water, dried naturally, and characterized by SEM and XRD. The mineralization solution consisted of 18.2 mM CaCl₂, 2H₂O, 5.6 mM K₂HPO₄—3H₂O, 1 ppm NaF, 0.14 mM NaCl, 2 mM HCl, and 83 mM Tris, and a pH of the mineralization solution was adjusted with a 10 mM Tris buffer to 6.5±0.1.

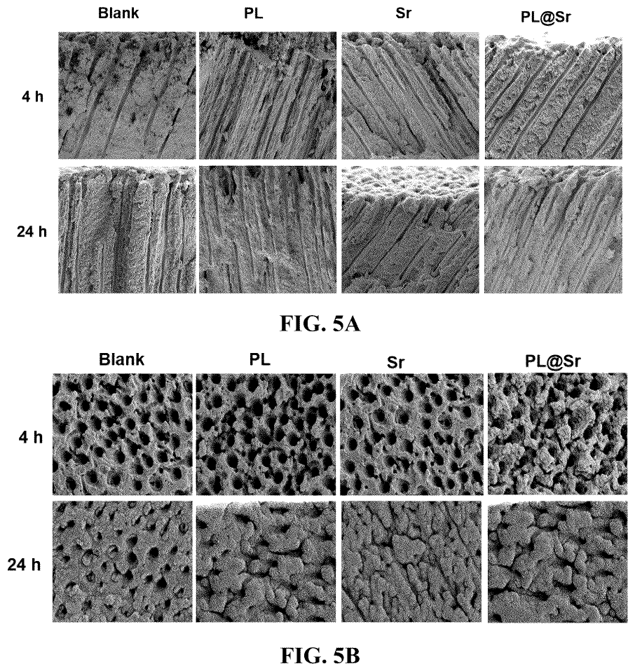

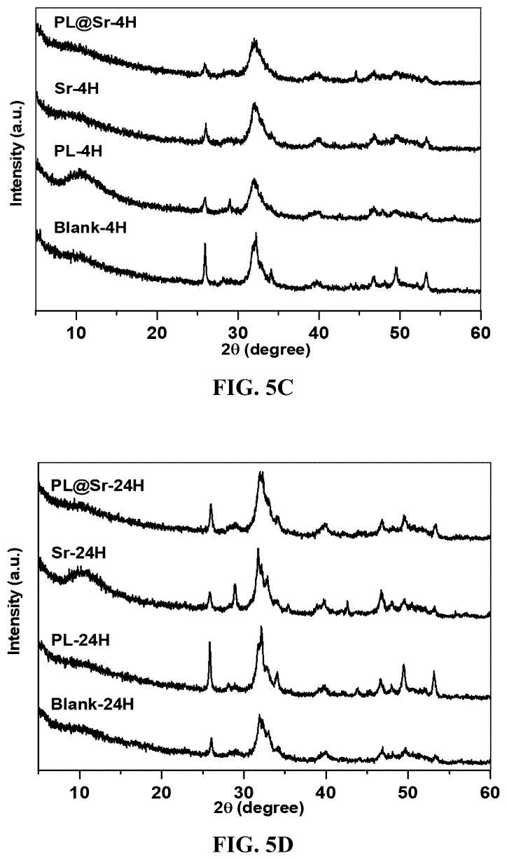

Surface SEM results of dentin of the four groups after 4 h and 24 h of remineralization were shown in FIG. 5A. It can be seen from FIG. 5B that diffraction peaks in the XRD patterns are consistent with those of the hydroxyapatite (JCPDS No.09-4032) card. The characteristic peaks of hydroxyapatite appear at 32.2°, 39.8°, and 46.7°, which correspond to the (002), (211), (112), (310), and (222) crystal planes, respectively.

### 5. Acid Corrosion Resistance Testing for Remineralized Dentin:

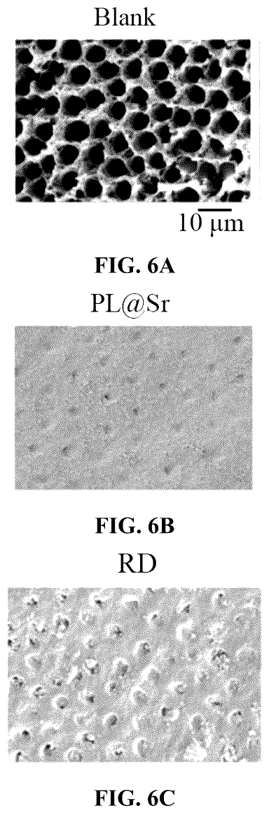

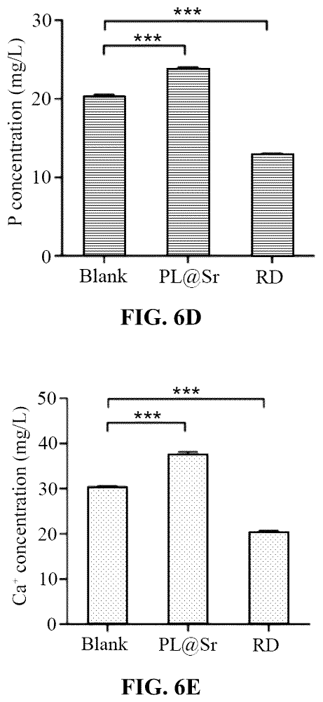

Mineralized dentin sections were soaked in a 5% citric acid solution for 90 s to evaluate the acid resistance. The remineralized dentin was coated with nail polish (candyMoyo, China) (24 h) to seal dentin outside the remineralized surface (exposed surface area: 4 mm×4 mm). The remineralized dentin was soaked in 2 mL of a 5% citric acid solution for 90 s. After the acid corrosion, the dentin was immersed in an Eppendorf tube with 1 mL of deionized water for 2 min to remove the residual calcium and phosphorus ions, and then immersed in another Eppendorf tube with 1 mL of fresh deionized water for 2 min. 2 mL of the citric acid solution and 2 mL of the deionized water after soaking were collected and tested for calcium and phosphorus ion concentrations by inductively coupled plasma optical emission spectrometry (ICP-oes). A surface morphology of dentin undergoing acid corrosion was observed by SEM. The healthy dentin and the PL@Sr dentin were taken as control groups. Results are shown in FIG. 6A to FIG. 6E.

FIG. 6A to FIG. 6C show the SEM surface morphologies of the normal dentin, the dentin sealed with PL@Sr, and the sealed dentin after mineralization for 24 h and corrosion in 5 wt % citric acid for 90 s. It can be seen from FIG. 6A to FIG. 6C that the sealed and mineralized dentin is obviously corroded. It can be seen from FIG. 6D and FIG. 6E that the sealed and mineralized dentin undergoes significant calcium and phosphorus losses after being corroded in citric acid for 90 s.

### 6. Frictional Wear Testing for Remineralized Dentin:

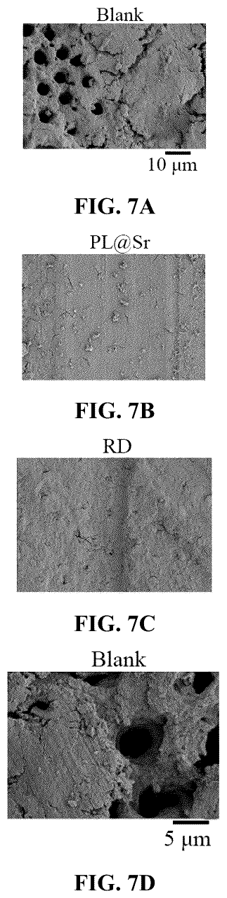

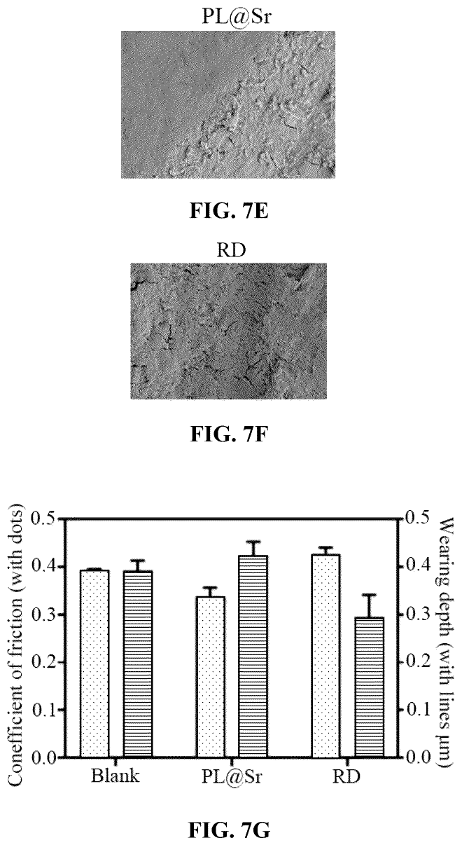

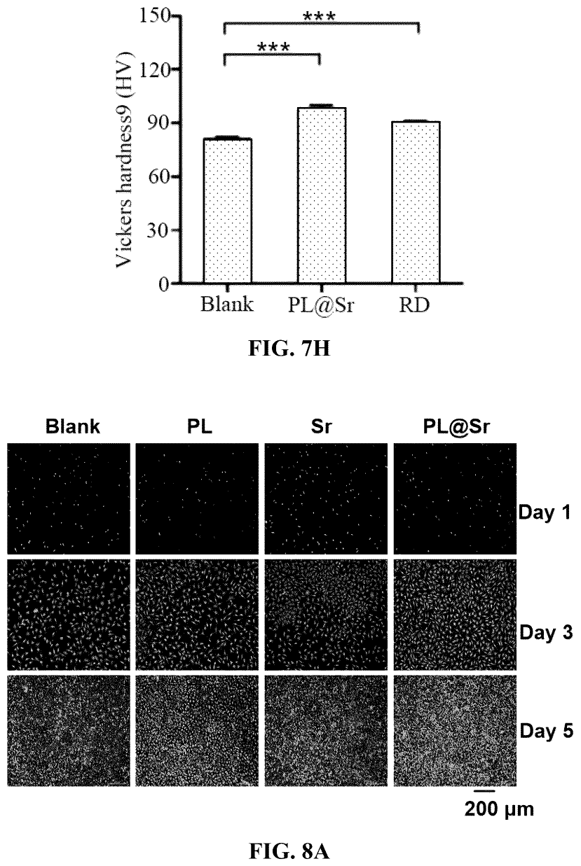

A frictional wear testing machine was used to measure the coefficients of friction and wearing depths of a remineralized dentin (24 h) group and a coating group. A reverse test was conducted using a 3 mm-long track under the following conditions: room temperature, a normal load of 1.5 N, a sliding speed of 1mm. s⁻¹, and a cycle of 30 s. The parameters such as coefficient of friction, z-axis, and z-depth were evaluated for each group (n=3). The wear traces of a sample after the backslip test were analyzed by field emission scanning electron microscopy. A coefficient of friction and a wearing depth were calculated with the RTEC Viewer software. The healthy dentin was adopted as a control group. Results are shown in FIG. 7A to FIG. 7H.

It can be seen from FIG. 7A to FIG. 7H that the basic tooth brushing with repeated rubbing does not have a significant impact on the PL@Sr-sealed dentin and the remineralization of dentin. In addition, the proper tooth brushing does not lead to the remineralization of a PL@Sr coating and the dentin.

### 7. Investigation of Biocompatibility of Different Components:

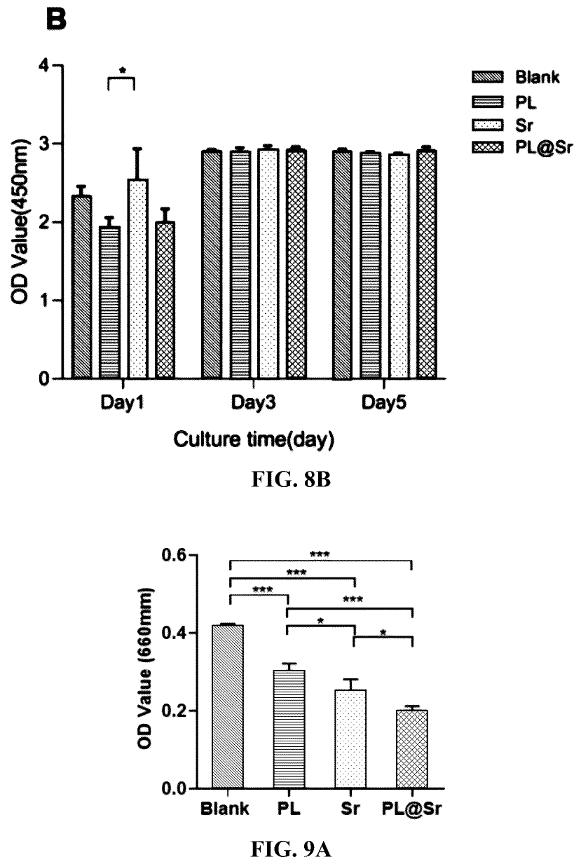

L929 was adopted as an experimental material for cell counting kit-8 (cck-8) assay. The number of cells growing was quantitatively detected. OD values on day 1, day 3, and day 5 were measured, respectively. Moreover, the fluorescent staining was conducted to observe the cell morphology. On day 1, day 3, and day 5 of culture, fluorescence images were acquired. Results are shown in FIG. 8A and FIG. 8B.

It can be seen from FIG. 8A and FIG. 8B that, on day 3 and day 5, there is no significant difference among OD values of the four groups (P>0.05), indicating that the PL@Sr group has excellent biocompatibility.

### 8. Evaluation of in Vitro Antibacterial Properties of Different Components:

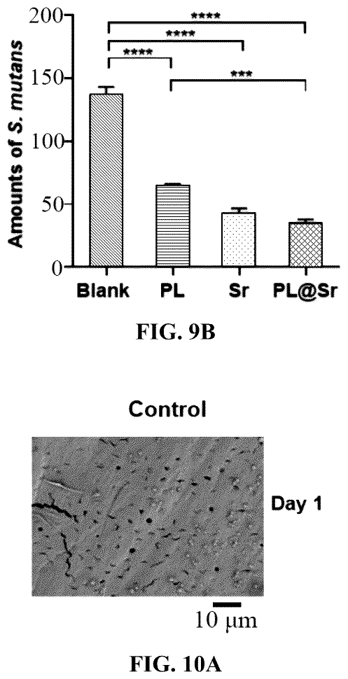

The anti-Streptococcus mutans performance of the prepared dentin sealing materials was evaluated by a turbidimetric method and a plate coating method. Results are shown in FIG. 9A and FIG. 9B.

It can be seen from FIG. 9A and FIG. 9B that the PL group, the Sr group, and the PL@Sr group all have an ability to inhibit Streptococcus mutans, and the PL@Sr group exhibits the optimal ability.

### 9. Animal Experiment:

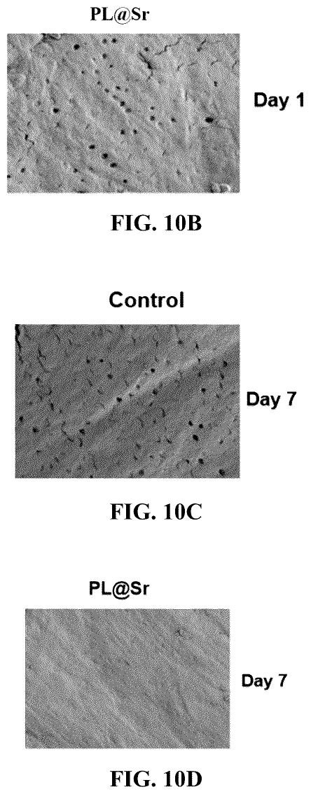

12 SD male rats that were about 4 weeks old and had a body weight of about 150 g were randomly divided into 4 groups (a blank group on day 1, a blank group on day 7, a PL@Sr group on day 1, and a PL@Sr group on day 7) with 3 rats in each group. The rats were intraperitoneally injected with 2.5% sodium pentobarbital to successfully establish in vivo models. After the anesthesia worked, the SD rats were fixed to lay flat, and first molar teeth at two sides of the upper jaw were exposed through a pulling hook and evenly rubbed with a ball drill for about 1 mm. In the PL@Sr group, tooth brushing was conducted twice a day, and administrated for 3 min each time. In the blank group, tooth brushing was conducted with normal saline twice a day. SD rats were sacrificed on day 1 and day 7. A sample was ultrasonically cleaned with normal saline for 60 s, taken out, and dried. A surface morphology of a first molar tooth of the upper jaw was observed by scanning electron microscopy. Results are shown in FIG. 10A to FIG. 10D.

It can be seen from FIG. 10A to FIG. 10D that PL@Sr exhibits a prominent DT-sealing effect in vivo. Although the present disclosure has been described in detail through the above embodiments, the embodiments are merely some rather than all the of the embodiments of the present disclosure. Other embodiments could be acquired based on these embodiments without creative efforts, and these embodiments shall fall within the scope of the present disclosure.

What is claimed is:

1. A complex for sealing a dentinal tubule (DT), wherein the complex is produced through polymerization of a catecholamine with metal ions,
wherein the catecholamine comprises one or more selected from the group consisting of dopamine, norepinephrine, tannic acid, gallic acid, phlorotannin (PL), phloroglucinol, and epigallocatechin gallate; and the metal ions comprise one or more selected from the group consisting of V²⁺ ions, Cr²⁺ ions, Cr³⁺ ions, Mn²⁺ ions, Mn³⁺ ions, Fe²⁺ ions, Fe³⁺ ions, Co²⁺ ions, Ni²⁺ ions, Cu²⁺ ions, and Sr²⁺ ions.

2. The complex for sealing the DT as claimed in claim 1, wherein the catecholamine is the PL, or
the metal ions are the Sr²⁺ ions.

3. (canceled)

4. A method for preparing the complex for sealing the DT as claimed in claim 1, comprising:
under aerobic and alkaline conditions, mixing the catecholamine and the metal ions to obtain a mixed solution, and subjecting the mixed solution to a reaction to produce the complex for sealing the DT.

5. The method as claimed in claim 4, wherein the reaction is conducted for 1 minute to 30 minutes, or
an alkaline condition refers to a pH value of 7.4 to 11.

6. (canceled)

7. The method as claimed in claim 4, wherein in the mixed solution obtained by mixing the catecholamine and the metal ions, a concentration of the catecholamine is in a range of 0.1 mg/mL to 5 mg/mL, and/or a concentration of the metal ions is in a range of 0.1 mg/mL to 5 mg/mL.

8. A reagent for treating dentin hypersensitivity (DH), comprising the complex for sealing the DT as claimed in claim 1.

9. The reagent for treating DH as claimed in claim 8, further comprising: an alkaline amino acid in a free or salt form, a fluoride salt, an anti-inflammatory compound, an antilithic agent, a surfactant, a humectant, an antibacterial agent, and an antioxidant, each in an effective amount.

10. The reagent for treating DH as claimed in claim 9, wherein the alkaline amino acid is one or more selected from the group consisting of arginine, lysine, citrulline, ornithine, creatine, histidine, diaminobutyric acid, diaminopropionic acid, and salts thereof.

11. The reagent for treating DH as claimed in claim 9, wherein the fluoride salt is one or more selected from the group consisting of stannous fluoride, sodium fluoride, potassium fluoride, sodium monofluorophosphate, sodium fluorosilicate, ammonium fluorosilicate, amine fluoride, ammonium fluoride, titanium fluoride, and a hexafluorosulfate salt.

12. The reagent for treating DH as claimed in claim 8, further comprising one or more selected from the group consisting of an abrasive and microparticles.

13. The reagent for treating DH as claimed in claim further comprising a whitening agent selected from the group consisting of whitening active substances.

14. The reagent for treating DH as claimed in claim 8, further comprising hydrogen peroxide or a hydrogen peroxide source, wherein the hydrogen peroxide source is selected from the group consisting of urea peroxide, a peroxide salt or a composite, and a hydrogen peroxide-polymer composite.

15. The reagent for treating DH as claimed in claim 9, further comprising a polymer, wherein the polymer is one or more selected from the group consisting of polyethylene glycol, a polymethyl vinyl ether-maleic acid copolymer, and a polysaccharide.

16. The reagent for treating DH as claimed in claim 9, further comprising a calcium and phosphate source, wherein the calcium and phosphate source is selected from the group consisting of a calcium-glass composite and a calcium-protein composite.

17. The reagent for treating DH as claimed in claim further comprising a soluble calcium salt.

18. The reagent for treating DH as claimed in claim 9, further comprising an agent for interfering with or preventing bacterial adhesion.

19. The reagent for treating DH as claimed in claim 9, further comprising a physiologically-acceptable potassium salt.

20. The reagent for treating DH as claimed in claim 9, wherein the reagent for treating DH is selected from the group consisting of a toothpaste, a mouthwash, a drug, a dental gel, a tooth powder, a non-abrasive gel, a mousse, a foam, an oral spray, a lozenge, an oral tablet, a dental tool, and a pet care product.

21. A method for treating a dental disease, comprising:
administrating the reagent for treating DH as claimed in claim 8 to a subject in need thereof,
wherein the dental disease is selected from the group consisting of DH, dental caries, and an enamel damage.

22-23. (canceled)

24. A toothpaste, comprising the reagent for treating DH as claimed in claim 8, and at least one item selected from the group consisting of water, an abrasive, a surfactant, a foaming agent, a vitamin, a polymer, an enzyme, a humectant, a thickening agent, an antibacterial agent, a preservative, a flavoring agent, and a coloring agent.
